(19) United States
(12) Patent Application Publication
(10) Pub. No.: US 2023/0324363 A1
(43) Pub. Date: Oct. 12, 2023

(54) DEVICE AND METHOD FOR DETECTING WATER FLOW THROUGH AT LEAST ONE LAYER OF BIOLOGICAL CELLS

(71) Applicants: FRAUNHOFER- GESELLSCHAFT ZUR FÖRDERUNG DER ANGEWANDTEN FORSCHUNG E.V., München (DE); UNIVERSITÄT REGENSBURG, Regensburg (DE)

(72) Inventors: Kathrin ZÖPFL, Regensburg (DE); Joachim WEGENER, München (DE); Florian URBAN, Regensburg (DE)

(73) Assignees: FRAUNHOFER-GESELLSCHAFT ZUR FÖRDERUNG DER ANGEWANDTEN FORSCHUNG E. V., München (DE); UNIVERSITÄT REGENSBURG, Regensburg (DE)

(21) Appl. No.: 18/042,478

(22) PCT Filed: Aug. 17, 2021
(86) PCT No.: PCT/EP2021/072849

§ 371 Date: Feb. 22, 2023

(30) Foreign Application Priority Data
Aug. 24, 2020 (DE) 10 2020 210 718.1

(51) Int. Cl. G01N 33/483; G01N 27/02

(52) U.S. Cl. CPC G01N 33/4833; G01N 27/026

(57) ABSTRACT

The invention relates to a device for detecting water flow through or over at least one layer of biological cells, the device containing a first and a second container, the second container being situated at least in part within the first container such that two separate compartments are formed. The base of the second container consists, at least in regions, of a planar, water-permeable substrate for growing biological cells, and the base of the first container has an electrode pair, the electrodes of which each have an electrical connection which leads into a space outside the first container. By means of the device according to the invention, water flow over at least one layer of biological cells can be detected easily, quickly, robustly, and reproducibly, with a high temporal resolution. The invention also relates to a method for detecting water flow over at least one layer of biological cells.

Provided is a device for detecting water transport through or via at least one layer of biological cells, the device comprising a first and a second container, the second container being situated at least in part within the first container such that two separate compartments are formed. The base of the second container consists, at least in regions, of a planar water-permeable substrate for growing biological cells, and the base of the first container has an electrode pair, the electrodes of which each have an electrical connection which leads into a space outside the first container. By means of the device according to the invention, water transport via at least one layer of biological cells can be detected easily, quickly, robustly, and reproducibly, with a high temporal resolution. The invention also relates to a method for detecting water transport via at least one layer of biological cells.

The assessment of the barrier function of cell clusters (for example tissue clusters) plays a fundamental role in the research and development of new medicaments, in particular in the targeted transport of medicaments to their place of action. Cell-based biomedical studies make use fundamentally of three parameters for quantitatively describing the barrier function of cell layers in cell clusters.

The first parameter is the permeability of the cell layer for molecular probes and is characterized by the so-called permeability coefficient (“P_{E} value”) (unit: in cm/s).

The second parameter is the electrical resistance of a cell layer and describes the permeability of the cell layer for inorganic ions (unit: Ω·cm²).

The third parameter is the permeability of the cell layer for water molecules and is generally described with the aid of the hydraulic conductivity L_{P} or the osmotic water permeability coefficient P_{OS} (unit: cm/s) depending on whether the water flow is induced by a hydrostatic gradient or an osmotic gradient along the cell layer.

To assess the water permeability of a cell layer, the focus is directed, due to the simpler experimental determinability, in particular to the water transport through the cell layer as the result of an osmotic gradient over the cell layer, which is quantified by the osmotic water permeability coefficient P_{OS}. High P_{OS} values here signify a particularly high water permeability of a cell layer. High P_{OS} values are obtained in particular for cell layers of kidney cells.

The degree of the water permeability of a cell layer is defined, in addition to the properties of the membrane lipids of the cell layer, in particular by certain water-permeable proteins (so-called water channels) in the cell membrane of individual cells of the cell layer. Known water channels are, for example, aquaporins. Depending on the number and subtype of aquaporins in the cell membrane of cells of a cell layer, the aquaporins change significantly the permeability of the cell layer for water. The biosynthesis of a certain number of aquaporins is therefore used by biological cells as a control element in order to adapt the water permeability of the cell membrane of the biological cells to their physiological requirements.

Aquaporin malfunctions, for example as the result of a corresponding gene mutation, lead to different illnesses, such as visual impairments, epileptic fits or obesity. Aquaporins are connected even with tumor growth and tumor spread in the body. The focus is thus also being directed increasingly towards aquaporins as target proteins (targets) for drug discovery for the treatment of these illnesses.

The functional screening of possible active ingredients for influencing the activity of aquaporins is, however, very difficult, because only inadequate measurement techniques for measuring the water permeability of cell layers have, to date, been available. The devices and methods known for this purpose in the prior art are not very robust, have a poor temporal resolution, do not allow lateral spatial resolution along the surface of a cell layer, and are unsuitable for a rapid examination of water transport through at least one layer of biological cells with medium to high throughput.

The water transport (or water flow) through a biological cell layer describes the water flow through the biological cell, i.e., through a first biological membrane on a first side of a biological cell (for example an upper side) into the biological cell and a water flow via at least one second biological membrane of the biological cell (for example an underside) from the biological cell, that is to say a water flow through at least two cell membranes. The volume of the biological cell does not necessarily change as a result of this water flow, since the outflow of water can be equal to or greater than the inflow of water.

The behavior is different, for example, when determining a transmembranal water transport (or water flow), which is not intended to be at issue here, but which will be described here briefly. The determination of the transmembranal water flow examines merely either the water transport from a cell into an extracellular space or from an extracellular space into a cell, i.e., with this type of water flow only a water flow through a single cell membrane is examined. Since the volume of the biological cells necessarily changes during this examination of the water flow through the inflow or outflow of water, the transmembranal water flow can be very easily determined by way of a volume change of the biological cells via the determination of a volume increase or volume decrease of the biological cells.

The determination of the water transport via a biological cell layer is more difficult, since for this purpose a determination solely of the volume increase or volume decrease of the cells is insufficient. The reason for this is that, during this examination of the water transport, the water may pass the cell such that there is no volume change at all of the biological cells of a cell layer (case: inflow of water=outflow of water). Another measurement technique is thus necessary for a detection of the water transport via a biological cell layer.

To determine the water transport via one or more biological cell layer(s), two devices or methods are known in principle in the prior art. In both variants the biological cells to be examined are firstly cultivated on a porous water-permeable substrate which is arranged in the base of a liquid container. Such a liquid container is often referred to as a “transwell insert”, wherein the porous, water-permeable substrate in the base of the container is often a porous polymer membrane. Due to the water permeability of the porous substrate on which the biological cells are cultivated prior to the measurement, the biological cells have access to an aqueous solution not only on their upper side, but also on their underside.

Following the cultivation on the porous, water-permeable substrate on the base of the liquid container, the liquid container is inserted into a second, larger liquid container and held by same such that the porous, water-permeable substrate has a certain distance from the base thereof. The used liquid container thus separates the resultant device into two liquid half-spaces, specifically one liquid half-space above the biological cell layer (upper or apical compartment) and one liquid half-space below the biological cell layer (lower or basal compartment). The porous, water-permeable substrate is designed according to number and size of the pores such that it does not influence considerably the measurement of the water permeability of a cell layer; the cell layer is intended to be the layer or entity between these two compartments that determines the speed for the water transport.

To determine the water permeability coefficient (P_{OS} value), it is known to add to one of the two liquid half-spaces a solution that is non-isotonic in respect of the other liquid half-space, in order to generate an osmotic gradient between upper and lower compartment and thus perpendicularly to the planar cell layer. The system attempts to compensate for this state of non-equilibrium by driving water in the direction of the higher osmolarity, wherein, in the case of a confluent cell layer, in which the cells are arranged gap-free next to one another on the porous, water-permeable substrate, the water must necessarily flow through the biological cells of the cell layer. The water permeability coefficient of the biological cell layer can be determined from the experimentally determined rate of this water transport through the biological cell layer.

In a first device known in the prior art or a first method known from the prior art, a thin glass capillary is used which is fluidically connected to one of the two liquid half-spaces. The water flow through the cell layer initiated by the osmotic gradient changes the liquid level in both compartments. For the compartment connected to the riser, the water flow can be quantified directly on the basis of the change to the liquid meniscus in the glass capillary. The exact meniscus level is generally analyzed in fully automated fashion by means of an electro-optical method. This technique is a direct measurement method, which, however, is technically complex and can only be parallelized with difficulty. For simultaneous testing of a plurality of cell layers under different conditions with medium to high throughput, such devices or methods are not economical and cannot be used cost-effectively. For example, in a parallel approach, a riser ought to be attached to each of the test devices, and each of these risers ought to be monitored in time-resolved fashion, which would require multiple electro-optical detection units. This technique is thus suitable more for basic research projects with low throughputs. For a high-throughout screening of large molecule libraries, for example for identifying modulators or aquaporins, these devices and methods are neither practicable nor economical.

In a second device known in the prior art or a second method known from the prior art, a non-cell-permeable fluorophore is introduced into one of the two compartments. Usually, FITC-, TRITC- or TexasRed™-labeled dextrans are used for this purpose in the relevant literature. The water flow through the cell layer results in either a dilution effect or a concentration effect of the fluorophore in the corresponding compartment and thus in changes of the fluorescence intensity over time. The change of the fluorescence intensity is measured by removal of a liquid sample from the compartment in which the fluorescence marker is located. The water amount transported into the compartment or out from the compartment can be determined from the temporal course of the fluorescence intensity and converted into P_{OS} values.

A disadvantage here is that the samples have to be removed at certain time intervals from the compartment to which the fluorophore has been added, which is time-consuming and can result in a loss of information between these time intervals, which results in a poor temporal resolution. In addition, the manual sample removal is susceptible to failure, whereby the reproducibility of the obtained results is low. Furthermore, the biological cell layer can be disturbed by the repeated manual removal of a sample, i.e., for example, the temperature, the pH value and/or the gas atmosphere in the measurement system can be changed, which can lead to significant artifacts and physiologically irrelevant measurement results. In addition, in this approach too, the possible sample throughput per time is heavily limited by the manual removal of samples. Furthermore, particularly with a high throughput, the costs for the use of the fluorophore may be high. Regardless of this, the presence of the fluorophore may in principle bring about its own biological effect, which may disturb the measurement result and in addition may also falsify the determination of the effect of a substance which was added in a targeted manner to the measurement system in order to examine its influence on the water transport of the cell layer.

The devices and methods known in the prior art additionally have the disadvantage that they cannot be used to detect the water transport via a cell layer in spatially resolved fashion along the lateral extent of the cell layer, and instead the water transport can only be detected integrally, i.e., ultimately a mean value of different local individual values of the water transport is determined. A spatially resolved determination of the water transport along the lateral extent of the cell layer, however, would be advantageous, since the water-barrier function of the cell layer is not necessarily distributed homogeneously over the entire surface of the cell layer. It would therefore be desirable to be able to test the water-barrier function of the entire surface of the cell layer at a plurality of points of the cell layer in spatially resolved fashion in order to obtain information about a lateral distribution of the water permeability coefficients along the cell layer. Furthermore, such a measurement would be less susceptible to faults or detects in the cell layer, since these could be easily identified via the spatial resolution and could be left out of consideration in the assessment.

To summarize, it is asserted that the prior art does not provide a device or method by which the water transport via a cell layer can be determined in a simple, quick, robust and reproducible manner with a high temporal and a high spatial resolution.

Proceeding from this, it was the object of the present invention to provide a device and a method for non-invasive detection of water transport via a cell layer which do not have the disadvantages from the prior art. In particular, it should be possible with the device and the method to determine water transport via a biological cell layer in a simple, quick, robust and reproducible manner with high temporal resolution. A high spatial resolution of the detection should preferably also be possible.

The object is achieved by the device having the features of claim 1, the method having the features of claim 12 and the use having the features of claim 16. The dependent claims describe advantageous developments.

According to the invention, a device is provided for detecting water transport through at least one layer of biological cells, the device having at least one detection chamber which
a) comprises at least one first container for receiving liquid; and
b) comprises at least one second container for receiving liquid which is arranged at least partially within the first container;
a base of the at least one second container consisting of a planar water-permeable substrate, the surfaces of this substrate being suitable for allowing biological cells to grow, and
the at least one second container being arranged in the at least one first container in such a way that a lower compartment is created in the at least one first container and an upper compartment is created in the at least one second container;
characterized in that a base of the at least one first container has at least one electrode pair, the electrodes of the at least one electrode pair each having an electrical connection which leads into a space outside the first container.

Within the scope of the present invention, the term “water transport” is understood to mean a “water flow”, i.e., the term “water transport” comprises a passive transport of water through the at least one layer of biological cells, which can also include a passive transport via water channels in the cell membranes of the biological cells.

The device according to the invention allows a non-invasive detection of water transport via at least one biological cell layer in a simple, quick, robust and reproducible manner with high time resolution. The temporal resolution of the examination possible with the device according to the invention lies in the region of a few seconds and consequently delivers precise information about the temporal course of the cellular water transport. In the device according to the invention, an electrical resistance can be measured (for example an impedance measurement) via an electrical contacting of the electrode pair or the device with use of low voltage amplitudes. In addition, the measurement is performed non-invasively, so that the examined cells are not influenced or damaged by the measurement and the cellular water transport can be observed also over a long measurement period without falsifying the measurement result.

The measurement with the device according to the invention can be performed fully computer-controlled and, following addition of an osmolyte to the first and/or second container, does not require any further interventions on the part of a user. A repeated removal of a sample, which is disruptive to the cell culture, with an inevitable associated disruption to the temperature and the gas and moisture atmosphere is unnecessary or is spared.

The device according to the preceding claim can be characterized in that the device has at least two, at least four, at least eight, at least 16, at least 32, at least 64 or at least 96 of these detection chambers, the detection chambers preferably being arranged next to one another on a plate and the respective electrical connections of the respective electrode pairs leading into a space outside the plate, in particular into a space below the plate. The plate can be embodied as a so-called well plate. The advantage of the plurality of detection chambers is that the water transport via at least one layer of biological cells can be measured in parallel (simultaneously), the biological layers possibly differing from detection chamber to detection chamber or the biological layers of the respective detection chambers possibly being the same and the respective detection chambers differing in respect of the composition of the liquids in the first and/or second container (for example different active ingredients in the liquids). The device according to the invention thus allows a simultaneous (automated) measurement with high parallelization and thus a much higher sample throughput than with known devices. This embodiment is thus advantageous in particular for pharmaceutical studies and biomedical research, in which high sample throughputs are a key economical criterion.

Further, the device according to the invention can be characterized in that the base of the at least one first container has at least two, preferably at least three, particularly preferably at least four, very particularly preferably at least five, in particular at least six, optionally at least ten, electrode pairs, the electrodes of the respective electrode pairs each having an electrical connection that leads into a space outside the first container. It is advantageous here that a spatially resolved detection of the water transport through the at least one layer of biological cells is possible, i.e., the water transport can be resolved in the lateral direction along the layer, whereby local differences in the water transport along the lateral extent of the layer can be discovered. This makes it possible to draw conclusions as to the homogeneity of the at least one layer of biological cells in respect of the water permeability properties and additionally allows statistically better verified results, since defects in the layer (for example holes in an otherwise confluent layer) can be discovered and excluded from the consideration.

In addition, the device according to the invention can be characterized in that the lower compartment has a further electrode, preferably a planar film electrode, and the upper compartment has a further electrode, preferably a stem electrode, the further electrode of the lower compartment preferably being arranged on the base of the at least one first container and/or the further electrode of the upper compartment preferably being held via a holder in the upper compartment or being arranged on an inner side wall of the at least one second container. The further electrodes in the upper and lower compartment have in particular an electrical connection, via which they can be electrically connected to a measuring device. This embodiment has the advantage that, besides the water transport through the at least one layer of biological cells, the transepithelial electrical resistance (TER) over the at least one layer of biological cells can also be determined. In contrast to devices from the prior art, there is thus no need for two independent devices or measurements in order to determine the degree of water transport (P_{OS} value) and the transepithelial electrical resistance (TER value). Both the TER value and the P_{OS} value can be determined via a measurement of the electrical conductivity (preferably an electrical impedance). The TER value delivers (integral) information about the permeability of the cell layer in respect of inorganic ions, whereas the P_{OS} value allows (also spatially resolved) information about the water permeability of the cell layer. Both parameters can be obtained from the same at least one layer of biological layers and thus can be directly correlated with one another.

The at least one first container of the device according to the invention is preferably dimensionally stable up to a temperature of 120° C. The advantage here is that the at least one first container of the device can be autoclaved, i.e., can be sterilized by temperature effect, without the first container being structurally damaged by this treatment. Further, this temperature stability allows the electrode pair of the at least one first container to be attached to the first container via a photolithographic structuring.

The at least one first container of the device can comprise or consist of plastic and/or glass, the plastic preferably being selected from the group consisting of polycarbonate, polyethylene terephthalate, polystyrene, poly(methyl methacrylate), polytetrafluoroethylene, polyurethane and mixtures and combinations thereof. The same can apply, similarly, for the at least one second container. In addition, the at least one second container can be embodied as disposable material. The advantage here is that the at least one second container can be disposed of following a measurement and does not have to be sterilized for a further measurement.

The at least one electrode pair on the base of the first container preferably comprises or consists of two film electrodes. The two film electrodes are preferably coplanar. The two film electrodes can be substantially round. Further, the two film electrodes can each have (on a side directed to the second container) an area in the range of from 0.01 to 5.0 mm², preferably 0.02 bis 1.0 mm², particularly preferably 0.05 bis 0.50 mm², in particular 0.10 to 0.25 mm². Regardless of this, the two film electrodes, at their closest point to one another, have a spacing from one another that lies in the range of from 50 to 1000 μm, preferably 100 to 600 μm, particularly preferably 200 to 400 μm, in particular 300 μm.

The device according to the invention can also be characterized in that the at least one electrode pair on the base of the first container is dimensionally stable up to a temperature of 120° C. The advantage here is that the first container can be autoclaved, i.e. can be sterilized by temperature effect, without the at least one electrode pair of the first container being damaged by this treatment. The at least one electrode pair can have an electrical resistance of at most 100 Ω/square. Further, the at least one electrode pair can comprise or consist of a material which is selected from the group consisting of metal, electrically conductive metal compound, carbon, electrically conductive plastic and combinations thereof, the material particularly preferably being selected from the group consisting of gold, titanium, indium-tin-oxide, graphene, polyaniline, polypyrrole, polythiophene, PEDOT and combinations thereof, the material being optionally doped and/or chemically modified.

The electrical connection of the electrodes of the at least one electrode pair, which leads into a space outside the first container, can comprise or consist of an electrical cable. Further, the electrical connection can be dimensionally stable up to a temperature of 120° C. The advantage here is that the first container can be autoclaved, i.e. can be sterilized by temperature effect, without the electrical connection of the at least one electrode pair of the first container being damaged by this treatment. The electrical connection preferably has an electrical resistance of at most 100 Ω/square. Further, the electrical connection can comprise or consist of a material which is selected from the group consisting of metal, electrically conductive metal compound, carbon, electrically conductive plastic and combinations thereof, the material particularly preferably being selected from the group consisting of gold, titanium, indium-tin-oxide, graphene, polyaniline, polypyrrole, polythiophene, PEDOT and combinations thereof, the material being optionally doped and/or chemically modified. The electrically conductive connection can additionally have an electrical insulation formed from an electrically non-conductive material, preferably embodied as a casing or coating or via an electrical line. In a preferred embodiment the electrical connection is electrically conductively connected to a device for measuring an electrical conductivity, the device preferably being configured to measure an electrical impedance.

The planar water-permeable substrate of the second container can comprise or consist of plastic and/or glass, the plastic particularly preferably being selected from the group consisting of polycarbonate, polyester, polyethylene terephthalate, polystyrene, poly(methyl methacrylate), collagen-coated polytetrafluoroethylene, polyurethane and mixtures and combinations thereof, the plastic being in particular polycarbonate.

Regardless of this, the planar water-permeable substrate of the second container has continuous pores with a mean pore diameter in the range of from 0.1 to 10.0 μm, preferably 0.2 to 7.5 μm, particularly preferably 0.4 to 5.0 μm, optionally 0.4 to 3.0 μm. The advantage of these pore diameters is that on the one hand water can flow through the pores and on the other hand the biological cells can be retained, i.e., remain on the substrate during the water transport.

In addition, the planar water-permeable substrate of the second container can have continuous pores with a pore density of at least 10⁵ pores per cm², preferably at least 10⁶ pores per cm², particularly preferably 10⁶ to 10⁸ pores per cm². Higher pore densities are advantageous since they allow a more exact detection of the water transport through the at least one layer of biological cells and in addition during the spatially resolved detection of the water transport in the lateral direction along the at least one layer of biological cells allow a finer spatial resolution.

The planar water-permeable substrate of the second container, perpendicularly to its planar extent, can further have a height in the range of from 5 to 100 μm, preferably in the range of from 10 to 50 μm, particularly preferably 15 to 30 μm. This height range on the one hand ensures sufficiently high mechanical stability in order to support the at least one layer of biological cells, and on the other hand forms only a short transport path for water through the substrate. The latter makes it possible to detect time-dependent changes of the water transport with a high temporal resolution.

The planar water-permeable substrate of the second container can have at least one layer of biological cells on a side facing away from the first compartment and/or on a side facing the first compartment. This embodiment has the advantage that the detection of water transport through the at least one layer of biological cells can be started without having to grow the biological cells beforehand on the planar water-permeable substrate. The measurement can thus be started more quickly.

In a preferred embodiment the at least one layer of biological cells is confluent, i.e., it is at least one gap-free layer of biological cells. The at least one layer of biological cells can be an individual layer (monolayer) of biological cells or can be a plurality of layers of biological cells arranged one on top of the other (for example a biological tissue). Further, the at least one layer of cells can comprise or consist of cells that either have no water transport protein in their cell membrane or have at least one water transport protein in their cell membrane, the water transport protein being in particular at least one aquaporin. The advantage of the first case is that the water transport through the layer of biological cells can be detected independently of a water transport protein. The advantage of the second case is that the water transport through the layer of biological cells can be measured under the influence of the water transport protein. If both determinations are performed with an otherwise identical layer of biological cells, the quantitative contribution of the water transport protein to the water transport through the at least one layer of biological cells can be calculated in addition by the different of the two determined degrees of water transport.

The device can comprise a temperature-control unit which is configured to keep the temperature of the device constant, the temperature-control unit preferably being a (for example external) heating unit, in particular a heat cabinet. The advantage here is that the user does not require a further temperature-control unit in order to keep the temperature of the device constant.

According to the invention, a method is additionally presented for detecting water transport through at least one layer of biological cells, the method comprising the steps of
a) providing a device according to the invention, the planar water-permeable substrate of the second container having at least one layer of biological cells on a side facing away from the first compartment and/or on a side facing the first compartment;
b) electrically connecting the electrodes of the at least one electrode pair to a device for measuring an electrical conductivity (for example via an electrical connection of their electrical connection which leads into a space outside the first container, with a device for measuring an electrical conductivity);
c) filling a first aqueous solution, which has an electrical conductivity, into the first compartment, the electrical conductivity of the first aqueous solution preferably being at most 20 mS/cm;
d) filling a second aqueous solution, which has an electrical conductivity, into the second compartment, the second aqueous solution having an osmolarity identical to the first aqueous solution, and the electrical conductivity of the second aqueous solution preferably being at most 20 mS/cm;
e) measuring an electrical conductivity of the first aqueous solution over the course of time in order to obtain a baseline;
f) adding an osmolyte (which for example is present in a liquid) to the first and/or second aqueous solution so that the osmolarity of the first aqueous solution differs from the osmolarity of the second aqueous solution;
g) measuring an electrical conductivity of the first aqueous solution over the course of time;
h) determining a difference between the electrical conductivity and the obtained baseline over the course of time, information about the water transport through the at least one layer of biological cells being derived from the difference;
   in particular steps b) to d) being performed in any order and steps e) to h) being performed in the stated order.

The method can be characterized in that the device used in the method is configured to measure an electrical conductivity to measure an electrical impedance. In particular, a change to the electrical impedance over the course of time is measured in steps e) and g). The electrical impedance is particularly preferably measured at an AC voltage amplitude of 50 mV. Furthermore, the electrical impedance is preferably measured at an AC voltage frequency of ≥10⁴ Hz, preferably an AC voltage frequency in the range of 10⁴ to 10⁶ Hz, in particular 10⁵ Hz. Alternatively it is preferred that the electrical impedance is measured at an AC voltage frequency of <10⁴ Hz, preferably 0.1 to 10 Hz, in particular 1 Hz, when the first compartment is to be or is filled with an aqueous solution that comprises a redox pair, preferably K₂[Fe(CN)₆]|K₃[Fe(CN)₆].

The method can be characterized in that the first aqueous solution and/or the second aqueous solution comprises a buffer that has a buffer effect in the region of pH 7 to 8. Further, the first and/or second aqueous solution can comprise a salt, preferably NaCl and KCl. In addition, the first and/or second aqueous solution can have a temperature in the range of from >0° C. to 55° C., preferably a temperature in the range of from 10° C. to 45° C., particularly preferably a temperature in the range of from 30° C. bis 40° C., in particular a temperature of 37° C.

The different osmolarity of the second aqueous solution to the first aqueous solution is preferably established via a different concentration of a soluble molecule which is selected from the group consisting of uncharged, non-cell-membrane-permeable molecules, the soluble molecule being particularly preferably selected from the group consisting of disaccharides, oligosaccharides, polysaccharides and combinations thereof. In particular, the soluble molecule is selected from the group consisting of sucrose, mannitol, inulin and combinations thereof.

The method can be characterized in that the first and/or the second aqueous solution comprises an active ingredient which is assumed to influence the water transport through the at least one layer of biological cells. This is preferably an active ingredient which is assumed to influence the activity of a water transport protein, in particular an aquaporin, in the cell membrane of the biological cells. For example, the discovery of selective and non-toxic aquaporin inhibitors in biomedical research is attributed great importance. It is presumed that inhibitors of the water channel AQP1 can limit the tumor spread and the tumor growth and that inhibitors against AQP4 can prevent brain swelling caused by strokes. The embodiment of the method described here can significantly facilitate and improve the efficiency of the identification of new and improved aquaporin inhibitors.

According to the invention, the use of a measurement of an electrical conductivity, preferably an impedimetric measurement, to detect water transport via at least one layer of biological cells is additionally proposed.

The subject matter in accordance with the invention will be explained in more detail with reference to the following Figures and Examples without intending to restrict it to the specific embodiments shown here.

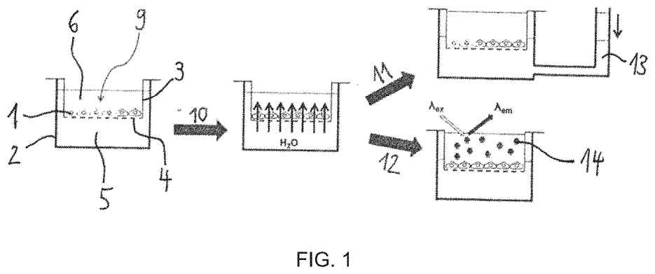

FIG. 1 shows a schematic depiction of a device for detecting osmotically induced water transport via a confluent individual layer of biological cells from the prior art. Biological cells are cultivated on the planar water-permeable substrate of the second container (here a “Transwell Insert” from the company Corning) until a confluent individual layer of the biological cells has formed. The second container is then transferred into the first container of the device, whereby two separate liquid compartments are created, i.e., one upper and one lower compartment. The osmolarity of the aqueous solution in the upper compartment (i.e., above the layer of the biological cells) is increased relative to the osmolarity of the aqueous solution in the lower compartment (for example by addition of the above-mentioned osmolytes, such as sucrose, mannitol or innulin). A water flow is induced from the lower to the upper compartment by the osmotic gradient between the upper and lower compartment and must necessarily pass through the individual layer of the biological cells. The degree of the water flow over the course of time is determined in the devices from the prior art either via a measurement of the change of the water level in a riser over the course of time (FIG. 1, top right) or via measurement of the change of a fluorescence intensity of a fluorescence dye in the aqueous solution in the upper compartment (FIG. 1, bottom right). The determined degree of the water flow over the course of time provides information about the water transport coefficient of the individual layer of biological cells.

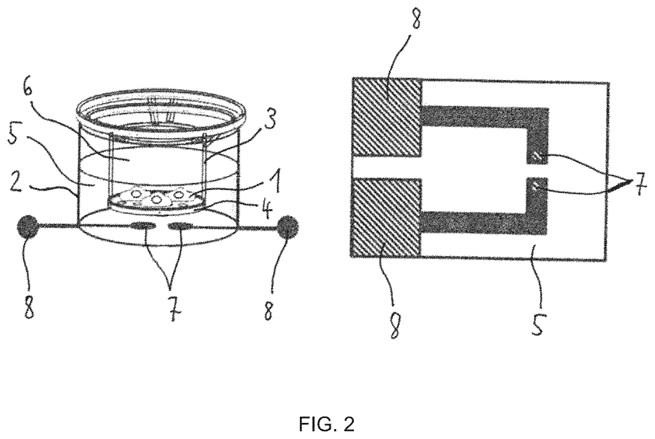

FIG. 2 shows schematically on the left a device according to the invention for impedimetric detection of osmotically induced water transport via a confluent individual layer of biological cells 4. The structure of the device is identical to FIG. 1, with the difference that the first compartment 5 does not need to have a fluidic connection to a riser and there is no need for a fluorescence marker to be present in the second compartment 6, and the base of the container, which forms the first compartment, has at least one electrode pair 7, the electrodes of the at least one electrode pair 7 each having an electrical connection 8 which leads into a space outside the first container 5. These electrical connections 8 are electrically conductively connected to a device for determining the electrical conductivity (for example an impedance measuring device, not shown). FIG. 2 shows on the right an alternative embodiment, in which the electrical connections 8 outside the first container 5 are formed by two large-area, square film electrodes 8 (shown by hatching on the right in FIG. 2). The two square film electrodes 8 are electrically conductively connected via electrical feed lines (shown in black) to the electrode pair 7, which is formed here by two small-area, coplanar and circular (d=500 μm) film electrodes (shown by hatching), which are arranged on the base of the first container 5. The feed lines are covered by an electrically insulating photopolymer, whereas the two square film electrodes 8 and the two circular electrodes of the electrode pair 7 are uncovered. The distance between the two center points of the electrodes of the electrode pair 7 is in this embodiment 800 μm, and the spacing at their closest points to one another is 300 μm.

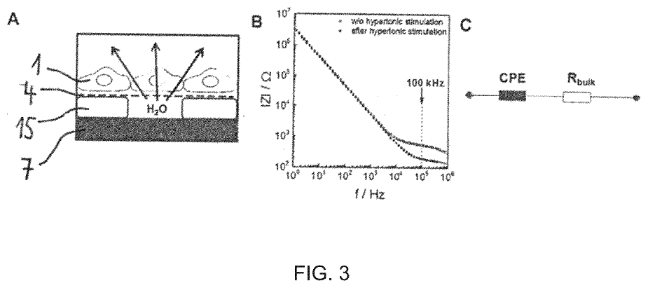

FIG. 3A shows schematically a detail of a side view of a device according to the invention. An electrode of the electrode pair (gold layer) is shown, which in the direction of the planar water-permeable substrate is covered in some regions with an electrically insulating photopolymer. FIG. 3B shows a typical impedance spectrum which can be obtained with a device according to the invention. The frequency range sensitive for determination of the water flow was in this case between 10⁴-10⁶ Hz, in which the overall impedance of the system is dominated by the electrolyte resistance. It can be seen that the osmotically induced water flow changes the electrical resistance of the solution in the first compartment. The best-suited detection frequency in the embodiment described here proved to be 10⁵ Hz (100 kHz). FIG. 3C describes the impedance spectrum shown at the bottom of FIG. 3B with the aid of an electrical equivalent circuit diagram. The electrode-electrolyte interface (1-10⁴ Hz) is described by a so-called constant phase element (CPE). The resistance of the aqueous solution in the first compartment (R_{bulk}) dominates the spectrum in the frequency range 10⁴-10⁶ Hz. The two spectra laid one above the other show the change in the frequency-dependent impedance by an osmotically induced water flow from bottom to top.

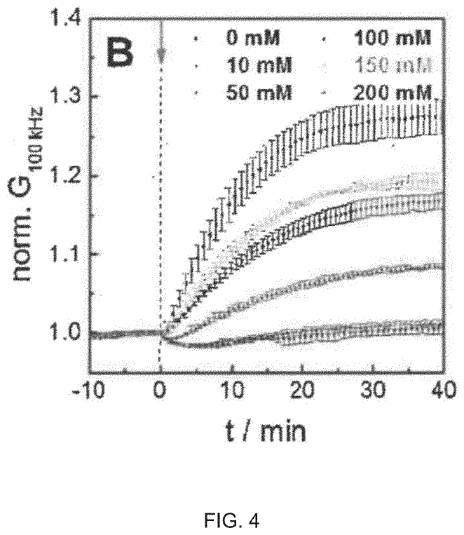

FIG. 4 shows the result of an experiment in which a confluent individual layer of the epithelial cell line MDCK-II is arranged on the planar water-permeable substrate, whereas the second aqueous solution in the upper compartment of the device has been selected to be hyperosmolar in comparison to the first aqueous solution in the lower compartment of the device. The hyperosmolarity in the upper compartment was generated here by adding sucrose to the aqueous solution of the upper compartment and leads to a water flow from the lower compartment to the upper compartment, as a result of which the conductivity in the lower compartment in the lower compartment rises as a function of time. The time-dependent change of the conductivity at a measurement frequency of 100 kHz in the lower compartment is shown and starts at t=0 min following establishment of the hyperosmolarity.

### EXAMPLE 1— METHOD FOR DETECTING WATER TRANSPORT THROUGH A CELL LAYER AT A MEASUREMENT FREQUENCY F OF >10⁴ HZ

The device according to the invention presented here by way of example has an electrode pair in a central position on the base of the chamber. In the exemplary embodiment described here, the coplanar electrodes of the electrode pair are manufactured from sputtered gold films with subsequent lithographic structuring. Gold is chemically inert, biocompatible, very electrically conductive, and the interface impedance corresponds in good approximation to an ideally polarizable electrode. The electrode structure consists of two equally sized, coplanar, circular film electrodes (d=500 μm), which are located on the base of the measuring device, centrally below the cell layer.

The feed lines, which are also made of gold, are coated with an insulating polymer.

The lithographically formed recesses in the polymer define the electrochemically active electrodes. The electrical connection of the electrodes to the measurement electronics (impedance analyzer) is made possible with the aid of two rectangular contact portions made of gold (cf. FIG. 2, right-hand side).

The measurement chamber is defined by a cell-compatible boundary (for example glass ring with d=2 cm), which is glued to the carrier substrate.

The impedance analysis is performed for example with the aid of a commercially conventional impedance analyzer.

Before the measurement is performed, at least one layer of biological cells is grown on a surface of the planar water-permeable substrate of the second container. The planar water-permeable substrate serves here as mechanical support for the biological cell layer. In the device according to the invention there is now a liquid half-space above the cell layer (upper compartment) and a liquid half-space below the cell layer (lower compartment).

The second container and porous polymer membrane thereof enclose, with the electrode pair on the base of the first container, a small liquid compartment, the exact volume of which is defined by the distance of the porous polymer membrane from the surface of the electrode pair, the spacing of the electrode pair and the thickness of the insulating polymer film applied to the electrical connections.

To carry out the measurement, the lower compartment comprising the electrode structure is filled with a first aqueous solution and the upper compartment is filled with a second aqueous solution, the first and second aqueous solutions being initially isotonic, i.e., having the same osmolarity. An electrical conductivity of the first aqueous solution is then measured over the course of time in order to obtain a baseline.

Once the baseline has been obtained, an osmolyte (for example in an aqueous liquid) is added to the first and/or second aqueous solution so that the osmolarity of the first aqueous solution differs from the osmolarity of the second aqueous solution. This measure induces water transport through the biological cell layer.

With osmotic stimulation of the cells by adding sucrose or mannitol to the upper compartment above the cell layer, water transport is induced through the cell layer from bottom to top. The electrolyte concentration in the lower compartment below the filter thus increases, and thus its conductivity, so that, after recording the baseline and performing a corresponding calibration of the electrical conductivity, it is possible to directly determine the effective water flow on the basis of the time-dependent measurement values (see FIG. 3A).

The electrolyte composition changed by an osmotically induced water flow thus leads to a corresponding change of the electrical resistance between the two electrodes of the electrode pair, which can be recorded with the aid of the measurement electronics. Consequently, the electrical conductivity (for example the electrical impedance) of the first aqueous solution is measured over the course of time in order to obtain a quantitative measure for the degree of the water flow. On the basis of the temporal changes of the electrical conductivity (for example impedance), it is possible to determine directly, i.e., for example without the use of a fluorophore as label, the water amount transported, and the water permeability coefficient, for the examined cell layer with a high temporal resolution.

In the case of the impedimetric detection principle, the frequency of the applied alternating voltage varies and the alternating current resistance (impedance) between the two coplanar electrodes of the electrode pair is determined as a function of this frequency (see FIG. 3B). The resultant impedance spectrum can be described with the aid of an electrochemical model (see FIG. 3C). The medium- to low-frequency range (1-10⁴ Hz) is characterized in the case of double-logarithmic plotting by an impedance that rises linearly with decreasing frequency and that is dominated by the electrode-electrolyte interface. This portion of the spectrum can be described by a so-called constant phase element (=CPE). The high-frequency range (10⁴-10⁶ Hz) is dominated by the electrical resistance of the aqueous solution in the first compartment (R_{bulk}) and can be used to determine the water flow. Consequently, the water flow via a cell layer can be detected by the associated change in the resistance or the conductivity in this frequency range.

In this embodiment the change of conductivity, that is to say the change of the reciprocal value of the real part of the impedance |Z| at a frequency of 100 kHz, serves as the best-suited readout parameter. An amplitude of the AC voltage smaller than 50 mV (pp) guarantees that the measurement itself has no influence on the cells located in the measuring device on porous polymer membranes. To convert the experimentally determined conductivity values into the electrolyte concentration and then from that into the amount of transported water, a calibration of the measurement set-up with standard solutions is performed.

The used standard solutions are differently concentrated KCl solutions, the individual conductivities of which have been determined independently with a standardized conductivity measuring cell. The cell constant determined in this way for the set-up described here makes it possible to convert any conductivity change into a change of the electrolyte concentration and thus to determine the amount of transported water. The time constant and the signal swing can be determined from the plotting of the measurement data (see FIG. 4) by adjustment of a saturation function. Both measurands can then be converted, following a published protocol, into the water permeability coefficients of the examined cell or tissue layer.

FIG. 4 shows the measurement data for a total of five increasingly hyperosmolar stimulations and the data of a control. The water permeability coefficient can be calculated from the time constants of the conductivity rise and the measured signal swing.

### EXAMPLE 2— METHOD FOR DETECTING WATER TRANSPORT VIA A CELL LAYER AT A MEASUREMENT FREQUENCY F OF <10 KHZ

This method described in Example 1 requires the measurement of the complex impedance at a measurement frequency of f>10 kHz (in Example 1, 100 kHz), the real part Re(Z) of which is then converted into the corresponding, time-dependent conductivity of the electrolyte.

In another embodiment of the method according to the invention the measurement is possible at a measurement frequency of less than 10 kHz.

The redox pair K₂[Fe(CN)₆]/K₃[Fe(CN)₆] is added to the buffer of the lower compartment in equimolar amounts, for example in a concentration of 1 mM, and the electrodes necessary for the impedance measurement are manufactured from gold films, for example with a thickness of 100 nm.

The presence of the redox pair induces a charge transfer via the otherwise ideally polarized electrode-electrolyte interface, which can be identified in the impedance spectrum by a flattening of the curve towards lower frequencies and can be quantified. The spectrum shown in FIG. 2 would in this case not rise linearly towards smaller frequencies, but would transition into a horizontal (frequency-dependent impedance course).

The intersection point of the horizontal with the Y-axis is determined by the concentration of the redox pair and is referred to as a charge-transfer resistance R_{ct}. The concentration-dependent R_{ct} can be expressed advantageously by the directly correlated real part of the impedance Rₑ(Z) at a correspondingly low frequency (for example 1 Hz).

If the redox pair is present in the lower compartment and an osmotically induced water flow from bottom to top has been established, the volume concentration of the redox pair in the lower compartment increases and the directly correlated charge-transfer resistance R_{ct} decreases correspondingly.

Very similarly to the embodiment described in Example 1, the water flow through the cell layer can thus be determined via the addition of a redox mediator and the determination of the concentration-dependent charge-transfer resistances.

In contrast to the method described in Example 1, the readout is not performed here at high frequencies of >10 kHz (especially: 100 kHz), but instead at low frequencies of less than 10 kHz (especially: 1 Hz).

No interference between the redox pair and the examined cells was identified for the assay times required here or the concentrations of the redox pair K₂[Fe(CN)₆]|K₃[Fe(CN)₆].

### EXAMPLE 3—METHOD FOR DETECTING WATER TRANSPORT VIA A CELL LAYER AT A PLURALITY OF LOCATIONS OF THE CELL LAYER (SPATIALLY RESOLVED DETERMINATION)

In devices from the prior art, the water permeability coefficient can be quantified merely as an integral variable for a given cell layer, i.e., only an average of the water permeability coefficient over the entire lateral extent of the cell layer can be specified. A lateral spatial resolution along the cell layer, which may indicate a possible heterogeneity of the water permeability coefficient at various points of the cell layer, was not previously accessible experimentally.

The determination of the P_{OS}, however, is not limited to one electrode pair per cell layer, i.e., a plurality of electrode pairs (for example an electrode pair array) can also be placed below the cell layer, which allow a corresponding measurement at various positions below the cell layer and thus make a spatial resolution accessible.

If, for example, five electrode pairs independent of one another are arranged on the base of the first container, below the cell layer, the detection of the water flow can be measured (simultaneously) at five different points along the lateral extent of the cell layer, and thus a spatially resolved measurement of the water transport along the lateral extent of the cell layer can be performed.

The degree of the spatial resolution can be increased further still by a further increase of the number of electrode pairs on the base of the first container below the cell layer.

### EXAMPLE 4—METHOD FOR DETECTING WATER TRANSPORT AND A TRANSEPITHELIAL ELECTRICAL RESISTANCE (TER) VIA A CELL LAYER

The experimental determination of the transepithelial electrical resistance (TER), similarly to the determination of the water permeability coefficient P_{OS}, requires a confluent cell layer on the planar water-permeable substrate, such that two liquid half-spaces above and below the cell layer are created.

Two additional electrodes are now placed in these two liquid half-spaces, such that an impedance-spectroscopic measurement can be performed through the cell layer and the TER can be derived from this. For this purpose, a punch electrode can be immersed in the upper compartment and a planar film electrode can be attached to the base of the lower compartment.

Such an electrode configuration for measuring TER and P_{OS} can be produced without difficulty using the established structuring techniques. Parallel and quasi-simultaneous measurements of TER and P_{OS} are thus possible in one measurement set-up and allow the water permeability of a cell layer to be combined with its barrier function to inorganic ions.

### LIST OF REFERENCE SIGNS

1: layer of biological cells;
2: first container;
3: second container;
4: planar water-permeable substrate;
5: lower compartment;
6: upper compartment;
7: electrode pair (or part thereof);
8: electrical connection of the electrode pair;
9: osmolyte (for example sucrose);
10: osmotic stimulation;
11: measurement via glass capillary (according to the prior art);
12: measurement via fluorescence dye dilution (according to the prior art);
13: glass capillary;
14: fluorescence dye;
15: electrically insulating photopolymer.

1-16. (canceled)

17. A device for detecting water transport through at least one layer of biological cells, the device having at least one detection chamber which
(a) comprises at least one first container for receiving liquid; and
(b) comprises at least one second container for receiving liquid which is arranged at least partially within the first container;
wherein a base of the at least one second container consists at least in some regions of a planar water-permeable substrate, wherein the surface of the substrate is suitable for allowing biological cells to grow, and
the at least one second container is arranged in the at least one first container in such a way that a lower compartment is created in the at least one first container and an upper compartment is created in the at least one second container;
wherein a base of the at least one first container has at least one electrode pair, the electrodes of the at least one electrode pair each having an electrical connection which leads into a space outside the first container.

18. The device according to claim 17, wherein the device has at least two, at least four, at least eight, at least 16, at least 32, at least 64 or at least 96 of these detection chambers.

19. The device according to claim 18, wherein the detection chambers are arranged next to one another on a plate and the respective electrical connections of the respective electrode pairs leading into a space outside the plate.

20. The device according to claim 17, wherein the base of the at least one first container has at least two electrode pairs, the electrodes of the respective electrode pairs each having an electrical connection that leads into a space outside the first container.

21. The device according to claim 17, wherein the lower compartment has a further electrode, and the upper compartment has a further electrode.

22. The device according to claim 17, wherein the at least one first container
(i) is dimensionally stable up to a temperature of 120° C.; and/or
(ii) comprises plastic and/or glass.

23. The device according to claim 22, wherein the plastic is selected from the group consisting of polycarbonate, polyethylene terephthalate, polystyrene, poly(methyl methacrylate), polytetrafluoroethylene, polyurethane, and mixtures and combinations thereof.

24. The device according to claim 17, wherein the at least one electrode pair on the base of the first container comprises two film electrodes.

25. The device according to claim 24, wherein the two film electrodes
(i) are coplanar; and/or
(ii) are substantially round;
(iii) each has an area in the range of from 0.01 to 5.0 mm²; and/or
(iv) at their closest point to one another have a distance from one another which is in the range of from 50 to 1000 μm.

26. The device according to claim 17, wherein the at least one electrode pair on the base of the first container
(i) is dimensionally stable up to a temperature of 120° C.; and/or
(ii) has an electrical resistance of at most 100 Ω/square; and/or
(iii) comprises a material selected from the group consisting of metal, electrically conductive metal compound, carbon, electrically conductive plastic and combinations thereof.

27. The device according to claim 17, wherein the electrical connection of the electrodes of the at least one electrode pair
(i) is dimensionally stable up to a temperature of 120° C.; and/or
(ii) has an electrical resistance of at most 100 Ω/square; and/or
(iii) comprises a material selected from the group consisting of metal, electrically conductive metal compound, carbon, electrically conductive plastic and combinations thereof, and/or
(iv) is electrically conductively connected to a device for measuring an electrical conductivity.

28. The device according to claim 17, wherein the water-permeable substrate of the second container
(i) comprises plastic and/or glass; and/or
(ii) has continuous pores with a mean pore diameter in the range of from 0.1 to 10.0 μm; and/or
(iii) has continuous pores with a pore density of at least 10⁵ pores per cm²; and/or
(iv) perpendicularly to its planar extent has a height in the range of from 5 to 100 μm; and/or
(v) is formed as disposable material.

29. The device according to claim 17, wherein the planar water-permeable substrate of the second container has at least one layer of biological cells on a side facing away from the first compartment and/or on a side facing the first compartment.

30. The device according to claim 29, wherein the at least one layer of biological cells
(i) is confluent; and/or
(ii) is an individual layer of biological cells or consists of a plurality of layers, arranged one above the other, of biological cells; and/or
(iii) comprises cells that either have no water transport protein in their cell membrane or have at least one water transport protein in their cell membrane.

31. The device according to claim 17, wherein the device comprises a temperature-control unit which is configured to keep the temperature of the device constant.

32. A method for detecting water transport through at least one layer of biological cells, the method comprising the steps, in any order, of
(a) providing a device according to claim 17, the planar water-permeable substrate of the second container having at least one layer of biological cells on a side facing away from the first compartment and/or on a side facing the first compartment;
(b) electrically connecting the electrodes of the at least one electrode pair to a device for measuring an electrical conductivity;
(c) filling a first aqueous solution, which has an electrical conductivity, into the first compartment;
(d) filling a second aqueous solution, which has an electrical conductivity, into the second compartment, the second aqueous solution having an osmolarity identical to the first aqueous solution;
(e) measuring an electrical conductivity of the first aqueous solution over the course of time in order to obtain a baseline;
(f) adding an osmolyte to the first and/or second aqueous solution so that the osmolarity of the first aqueous solution differs from the osmolarity of the second aqueous solution;
(g) measuring an electrical conductivity of the first aqueous solution over the course of time; and
(h) determining a difference between the electrical conductivity and the obtained baseline over the course of time, information about the water transport through the at least one layer of biological cells being derived from the difference.

33. The method according to claim 32, wherein the device for measuring an electrical conductivity is configured to measure an electrical impedance.

34. The method according to claim 32, wherein the first aqueous solution and/or the second aqueous solution
(i) comprises a buffer that has a buffer effect in the range of from pH 7 to 8; and/or
(ii) comprises a salt; and/or
(iii) has a temperature in the range of from >0° C. to 55° C.

35. The method according to claim 32, wherein the first and/or second aqueous solution comprises an active ingredient which is assumed to influence the water transport via the at least one layer of biological cells.
